(19) United States
(12) Patent Application Publication
(10) Pub. No.: US 2026/0341537 A1
(43) Pub. Date: Oct. 29, 2026

(54) METHOD OF IDENTIFYING DRUGGABLE TARGET GENE

(71) Applicant: GRAPHEN DRUGOMICS, INC., New York, NY (US)

(72) Inventors: Sing-Han HUANG, Taipei City (TW); Tzu-Ying TU, Taipei City (TW); Yi-Ting CHEN, Taipei City (TW); Xiang-Yu LIN, Taipei City (TW); Ssu-Ting LIEN, Taipei City (TW); Chiao-Wen CHEN, Taipei City (TW); Cheng-Tang WANG, Taipei City (TW); Ching-Yung LIN, Scarsdale, NY (US)

(21) Appl. No.: 19/660,943

(22) Filed: Apr. 28, 2026

Related U.S. Application Data
(60) Provisional application No. 63/796,488, filed on Apr. 29, 2025.

(51) Int. Cl. G16H 70/40; G16B 25/00

(52) U.S. Cl. CPC G16H 70/40; G16B 25/00

(57) ABSTRACT

A method includes: establishing a target knowledge database based on multi-omics data; constructing a heterogeneous graph related to a specific disease; obtaining, for each of candidate genes, a gene-node contribution score by using the heterogeneous graph as an input to a heterogeneous graph transformer (HGT); generating, based on pieces of target knowledge data included in the target knowledge database and the gene-node contribution scores for the candidate genes, a target integrated evaluation table that contains comprehensive assessment scores related to the candidate genes; and selecting one of the candidate genes that corresponds to a greatest one of the comprehensive assessment scores in the target integrated evaluation table as a druggable target gene.

### CROSS-REFERENCE TO RELATED APPLICATION

This application claims the benefit of U.S. Provisional Patent Application No. 63/796,488, filed on Apr. 29, 2025, the entire disclosure of which is incorporated by reference herein.

### FIELD

The disclosure relates to a method of identifying a druggable target gene for a specific disease from among multiple candidate genes.

### BACKGROUND

A conventional method of identifying a druggable target gene (i.e., a gene that is critical to regulating onset and progression of a specific disease) relies heavily on single-omics data, such as data of differential gene expression (DGE) analysis, data of mutation frequency analysis, or data of citation frequency/count analysis. Consequently, it is difficult for the conventional method to take complex structures of biological interactions (e.g., drug-target interactions, biological signaling pathways involved in the specific disease) into account, causing insufficient precision and resulting in high false positive identification rates using the conventional method. Moreover, an artificial intelligence (AI) model used in the conventional method often lacks transparency and biological interpretability.

### SUMMARY

Therefore, an object of the disclosure is to provide a method of identifying a druggable target gene for a specific disease from among multiple candidate genes that can alleviate at least one of the drawbacks of the prior art.

According to the disclosure, the method includes steps of:
establishing, based on multi-omics data, a target knowledge database that includes multiple pieces of target knowledge data related respectively to the candidate genes;
constructing a heterogeneous graph related to the specific disease;
obtaining, for each of the candidate genes, a gene-node contribution score by using the heterogeneous graph as an input to a heterogeneous graph transformer (HGT);
generating, based on the pieces of target knowledge data included in the target knowledge database and the gene-node contribution scores respectively for the candidate genes, a target integrated evaluation table that contains multiple comprehensive assessment scores related respectively to the candidate genes; and
selecting one of the candidate genes that corresponds to a greatest one of the comprehensive assessment scores in the target integrated evaluation table as the druggable target gene.

### BRIEF DESCRIPTION OF THE DRAWINGS

Other features and advantages of the disclosure will become apparent in the following detailed description of the embodiment(s) with reference to the accompanying drawings. It is noted that various features may not be drawn to scale.

FIG. 1 is a block diagram illustrating a computing device according to an embodiment of the disclosure.

FIG. 2 is a flow chart illustrating a method of identifying a druggable target gene according to an embodiment of the disclosure.

FIG. 3 is a schematic diagram illustrating multiple pieces of target knowledge data included in a target knowledge database according to an embodiment of the disclosure.

FIG. 4 is a flow chart illustrating sub-steps of constructing a heterogeneous graph according to an embodiment of the disclosure.

FIG. 5 is a flow chart illustrating sub-steps of obtaining gene-node contribution scores according to an embodiment of the disclosure.

FIG. 6 is a flow chart illustrating a procedure of training a heterogeneous graph transformer (HGT) according to an embodiment of the disclosure.

### DETAILED DESCRIPTION

Before the disclosure is described in greater detail, it should be noted that where considered appropriate, reference numerals or terminal portions of reference numerals have been repeated among the figures to indicate corresponding or analogous elements, which may optionally have similar characteristics.

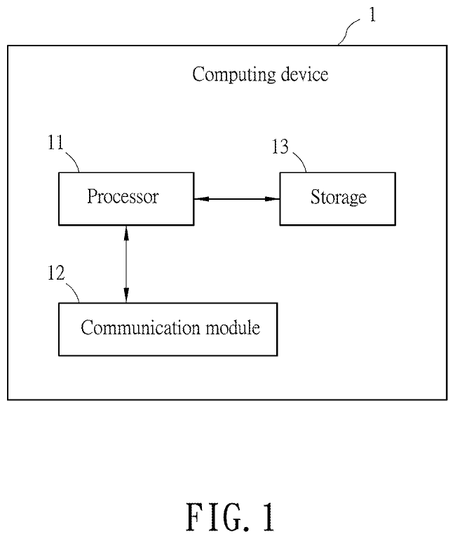

Referring to FIG. 1, an embodiment of a computing device 1 according to the disclosure is illustrated. The computing device 1 may be implemented to be a desktop computer, a laptop computer, a notebook computer, a tablet computer, a computing server or a data server, but implementation thereof is not limited to what are disclosed herein and may vary in other embodiments. The computing device 1 includes a communication module 12, a storage 13, and a processor 11 that is electrically connected to the communication module 12 and the storage 13.

The processor 11 may be implemented by a central processing unit (CPU), a microprocessor, a micro control unit (MCU), a system on a chip (SoC), or any circuit configurable/programmable in a software manner and/or hardware manner to implement functionalities discussed in this disclosure.

The communication module 12 may be implemented to be a network interface controller or a wireless transceiver that supports wireless communication standards, such as Bluetooth® technology standards, Wi-Fi technology standards and/or cellular network technology standards, but is not limited thereto.

The storage 13 may be implemented by random access memory (RAM), double data rate synchronous dynamic random access memory (DDR SDRAM), read only memory (ROM), programmable ROM (PROM), flash memory, a hard disk drive (HDD), a solid state disk (SSD), electrically-erasable programmable read-only memory (EEPROM) or any other volatile/non-volatile memory devices, but is not limited thereto. The storage 13 stores a heterogeneous graph transformer (HGT). Since implementation of the HGT has been well known to one skilled in the relevant art, detailed explanation of the same is omitted herein for the sake of brevity.

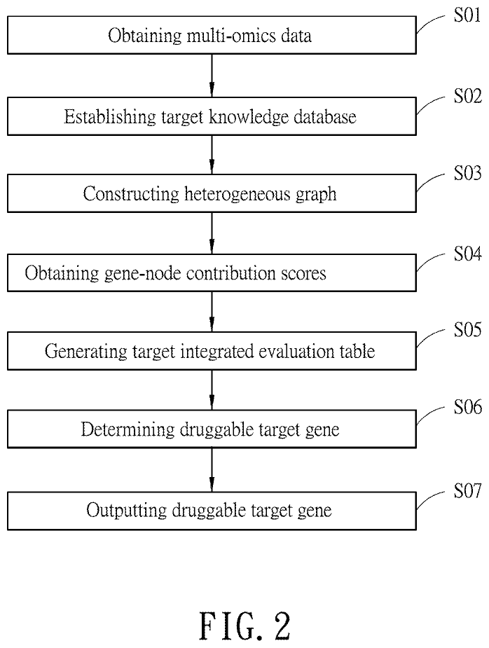

Referring to FIG. 2, an embodiment of a method of identifying a druggable target gene for a specific disease from among multiple candidate genes according to the disclosure is illustrated. It is worthy of note that a druggable target gene is a pivotal gene that is essential to regulating onset and progression of a specific disease. In this embodiment, the specific disease is kidney chromophobe renal cell carcinoma (KICH), but is not limited thereto. The candidate genes may be determined according to experience and expertise of technicians, or may include all known human genes currently. The method is to be implemented by the processor 11 of the computing device 1 that is previously described. The method includes steps S01 to S07 delineated below.

In step S01, the processor 11 obtains multi-omics data from multiple remote databases over communication networks (e.g., the Internet) via the communication module 12. The multi-omics data may include data from the Human Protein Atlas (THPA) portal database, the Open Targets Platform, the Kyoto Encyclopedia of Genes and Genomes (KEGG) database, the Reactome database, the Dependency Map (DepMap) Portal database, but is not limited thereto.

In step S02, the processor 11 establishes, based on the multi-omics data thus obtained, a target knowledge database that includes an M number of pieces of target knowledge data related respectively to the candidate genes, where M is a positive integer.

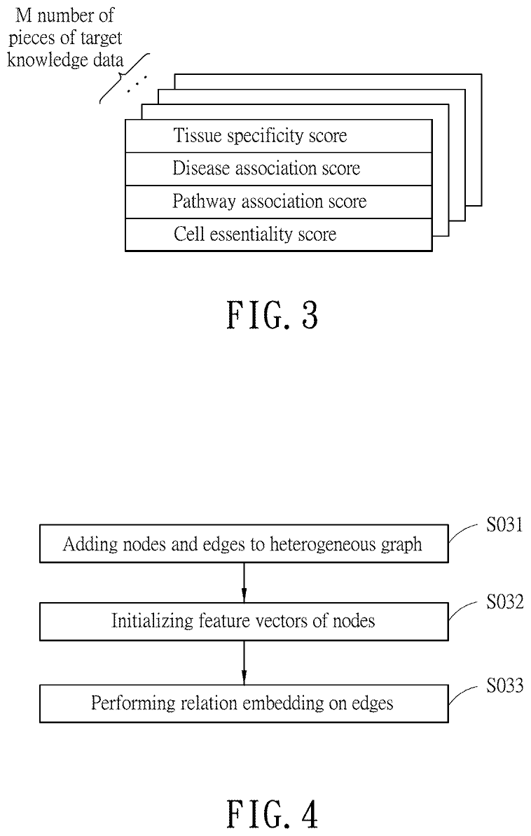

Referring to FIG. 3, each of the pieces of target knowledge data includes a tissue specificity score that indicates an expression distribution of a corresponding one of the candidate genes in a specific tissue, a disease association score that quantifies a level of association between the corresponding one of the candidate genes and the specific disease, a pathway association score that indicates an impact of the corresponding one of the candidate gene on specific biological signaling pathways, and a cell essentiality score that indicates an effect of inhibiting the corresponding one of the candidate genes on cell survival and cell proliferation. In particular, the tissue specificity score is obtained by performing standardization on a ratio of a local expression level of the corresponding one of the candidate genes in the specific tissue to an average expression level of the corresponding one of the candidate genes in all tissues. The tissue specificity score is obtained based on data from the THPA portal database. The disease association score is an association score between the corresponding one of the candidate genes and the specific disease obtained from the Open Targets Platform. The pathway association score is obtained by determining multiple specific biological signaling pathways that are related to the corresponding one of the candidate genes, performing enrichment analysis on the specific biological signaling pathways to obtain p-values respectively of the specific biological signaling pathways, converting the p-values respectively into z-values as participation scores of the corresponding one of the candidate genes, and calculating an arithmetic average of the participation scores of the candidate gene as the pathway association score of the corresponding one of the candidate genes. The pathway association score is obtained based on data from the KEGG database and/or the Reactome database. The cell essentiality score is obtained by multiplying a gene effect score from the DepMap Portal database by minus one.

In step S03, the processor 11 constructs a heterogeneous graph related to the specific disease. Specifically, step S03 includes sub-steps S031 to S033 as shown in FIG. 4 and delineated below.

In sub-step S031, the processor 11 adds multiple nodes respectively representing the candidate genes, multiple nodes respectively representing multiple candidate drugs for the specific disease, and multiple edges to the heterogeneous graph.

In sub-step S032, for each of the nodes that represent the candidate genes, the processor 11 initializes a feature vector of the node based on a piece of node-feature data related to a corresponding one of the candidate genes. The feature vector of the node that represents one of the candidate genes includes an expression level of a transcriptome related to the one of the candidate genes, a gene-mutation indicator indicating whether or not a gene mutation occurs on the one of the candidate genes, a DEG indicator indicating whether or not the one of the candidate genes is a differentially expressed gene (DEG), and a signaling-pathway indicator indicating whether or not the one of the candidate genes is involved in a biological signaling pathway relevant to the specific disease. In addition, for each of the nodes that represent the candidate drugs, the processor 11 initializes a feature vector of the node based on a piece of node-feature data related to the candidate drug. The feature vector of the node that represents one of the candidate drugs includes a chemical fingerprint that profiles compounds of the one the candidate drugs, and a therapeutic indicator indicating whether or not the one of the candidate drugs has a therapeutic effect on the specific disease. The piece of node-feature data related to each of the candidate genes and the piece of node-feature data related to each of the candidate drugs may be obtained from The Cancer Genome Atlas (TCGA) database, the Cancer Cell Line Encyclopedia (CCLE) database, the Gene Expression Omnibus (GEO) database, but is not limited thereto.

In sub-step S033, for each of the edges, the processor 11 performs relation embedding on the edge with a relational descriptor that is related to a function type based on a piece of edge-feature data that is related to a protein-protein interaction (PPI) or a drug-target interaction (DTI). The function type of the edge indicated by the relational descriptor is one of an adjustment (e.g., activation and inhibition), a physical action (e.g., binding) and a clinical effect (e.g., treatment). It is worthy of note that the piece of edge-feature data may be obtained from the integrative Biomedical Knowledge Hub (iBKH) database, but is not limited thereto.

In mathematics, a heterogeneous graph can be expressed as gₛ=(v_{g}, v_{d}, ε_{gg}, ε_{dg}), where v_{g} represents a set of all nodes that represents the candidate genes, v_{d} represents a set of all nodes that represents the candidate drugs, ε_{gg} represents a set of all edges each of which is between two nodes respectively representing two of the candidate genes, ε_{ag} represents a set of all edges each between two nodes one of which represents one of the candidate genes and the other of which represents one of the candidate drugs. For each node i that represents one of the candidate genes, i.e., iϵv_{g}, the feature vector of the node can be expressed as

$${x}_{i}^{g} = [ \begin{matrix}{x}_{i}^{expr} \\ {x}_{i}^{deg} \\ {x}_{i}^{mut} \\ {x}_{i}^{path}\end{matrix} ] ∈ {ℝ}^{4} ,$$

where

$${x}_{i}^{expr}$$

represents the expression level of a transcriptome related to the one of the candidate genes,

$${x}_{i}^{deg}$$

represents the DEG indicator,

$${x}_{i}^{mut}$$

represents the gene-mutation indicator, and

$${x}_{i}^{path}$$

represents the signaling-pathway indicator. For each node j that represents one of the candidate drugs, i.e., jϵv_{g}, the feature vector of the node can be expressed as

$${x}_{j}^{d} = [ \begin{matrix}{f}_{j}^{fp} \\ {x}_{j}^{dis}\end{matrix} ] ∈ {ℝ}^{{F}_{d}} ,$$

where

$${f}_{j}^{fp}$$

represents the chemical fingerprint, and

$${x}_{j}^{dis}$$

represents the therapeutic indicator. Each edge eᵢⱼ that has performed relation embedding with a relational descriptor rᵢⱼ can be expressed as eᵢⱼ=Embed(rᵢⱼ)ϵ [symbol] ^{d}^{ᵣ}, where Embed( ) is a function of relation embedding. Since the function of relation embedding has been well known to one skilled in the relevant art, detailed explanation of the same is omitted herein for the sake of brevity.

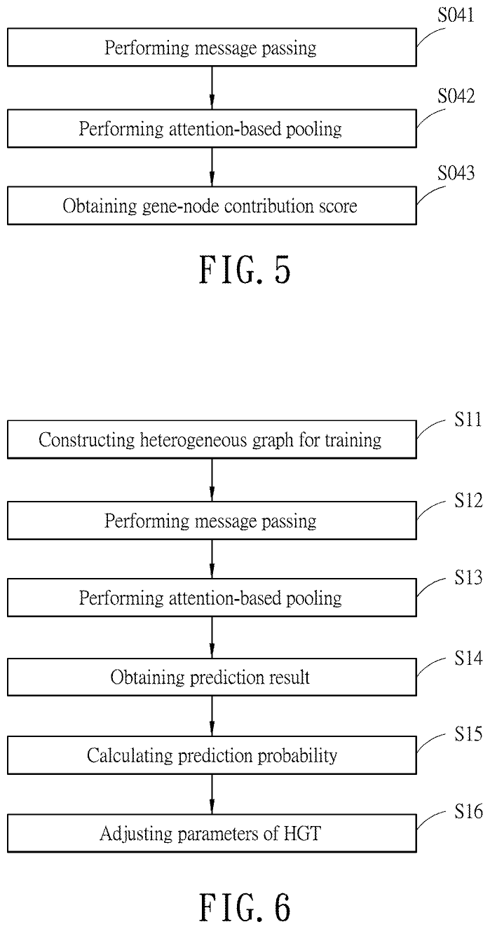

In step S04, the processor 11 obtains, for each of the candidate genes, a gene-node contribution score by using the heterogeneous graph as an input to the HGT stored in the storage 13. Specifically, step S04 includes sub-steps S041 to S043 as shown in FIG. 5 and delineated below.

In sub-step S041, the processor 11 performs, by using the HGT, message passing with multi-head attention to update the feature vectors respectively of the nodes of the heterogeneous graph.

In mathematics, for an edge that is between a current node i and a neighbor node j and that is embedded with a relational descriptor eᵢⱼ, with respect to an attention head h of the multi-head attention at a layer l of the HGT where each of i, j, h and l is an integer, several computations in the following are involved in message passing. The processor 11 calculates a query vector

$${q}_{i}^{h} = {W}_{Q}^{h} {x}_{i} ,$$

where

$${W}_{Q}^{h}$$

is a learnable linear weight matrix for the attention head h, and xᵢ is the feature vector of the current node i. The processor 11 calculates a key vector

$${k}_{j}^{h} = {W}_{K}^{h} {x}_{j} + {U}_{K}^{h} {e}_{ij ,}$$

where

$${W}_{K}^{h}$$

is a learnable linear weight matrix

$${U}_{K}^{h}$$

for the attention head h, xⱼ is the feature vector of the current node J, and is a learnable parameter linear weight matrix for the attention head h. The processor 11 calculates a value vector

$${v}_{j}^{h} = {W}_{V}^{h} {x}_{j} + {U}_{V}^{h} {e}_{ij} ,$$

where

$${W}_{V}^{h}$$

is a learnable linear weight matrix for the attention head h, and

$${U}_{V}^{h}$$

is a learnable parameter. The processor 11 calculates, by using a softmax function, an attention score

$${α}_{ij}^{h} = \frac{exp ( \frac{{( {q}_{i}^{h} )}^{T} {k}_{j}^{h}}{\sqrt{{d}_{k}}} )}{{\sum }_{j ∈ N ( i )} exp ( \frac{{( {q}_{i}^{h} )}^{T} {k}_{j}^{h}}{\sqrt{{d}_{k}}}} ,$$

where dₖ is a scaling factor for maintaining a sufficient amount of gradient. The processor 11 calculates, for the current node i for the attention head h, an information-aggregation vector

$${m}_{i}^{h} = {\sum }_{j ∈ N ( i )} {α}_{ij}^{h} {v}_{j}^{h} .$$

The processor 11 performs residual connection and layer normalization on

$${h}_{i}^{l} ,$$

which is an original feature vector of the current node i at the layer l of the HGT, to obtain

$${h}_{i}^{l + 1} = LayerNorm ( ReLU ( {m}_{i} ) + {W}_{res} {h}_{i}^{l} ) ,$$

which is an updated feature vector of the current node i at a layer l+1 of the HGT, where mᵢ is a result of residual connection performed for all attention heads, Wᵣₑₛ is a residual projection matrix, ReLU( ) is a rectified linear unit activation function, and LayerNorm( ) is a layer normalization function. Since the softmax function, the rectified linear unit activation function and the layer normalization function have been well known to one skilled in the relevant art, detailed explanation of the same is omitted herein for the sake of brevity.

In sub-step S042, after the feature vectors respectively of the nodes of the heterogeneous graph have been updated, the processor 11 performs attention-based pooling to integrate the feature vectors into a sample-level vector. Specifically, the processor 11 determines multiple attention weights respectively for the nodes of the heterogeneous graph, calculates multiple weighted vectors by multiplying the feature vectors respectively by the attention weights, and calculates an average of the weighted vectors as the sample-level vector. For one of the nodes representing the candidate genes, the corresponding one of the attention weights represents a significance of the corresponding one of the candidate genes to the specific disease. It is worthy of note that the sample-level vector is used for training the HGT, and represents a trend of an overall physiological condition (e.g., whether or not the specific disease occurs) of a sample (e.g., a person). In some embodiments, the processor 11 only determines the attention weights respectively for the nodes of the heterogeneous graph.

In mathematics, for a node i that represents one of the candidate genes, the attention weight gᵢ determined for the node i can be expressed as gᵢ=σ(w^{T}hᵢ), where hᵢ is a feature vector of the current node i, w^{T} is a transposed learnable weight vector of an attention gate of the HGT for scalar projection, and σ( ) is a nonlinear function for normalizing an input value to fall in a range from zero to one. The sample-level vector hₛ is calculated as

$${h}_{s} = \frac{{\sum }_{i ∈ {v}_{g}} {g}_{i} {h}_{i}}{{\sum }_{i ∈ {v}_{g}} {g}_{i}} .$$

In one embodiment, when the node i represents the DEG (i.e., the DEG indicator of the feature vector of the node i indicates that the node i is the DEG), the attention weight for the node i will be adjusted as

$${g}_{i}^{′} = {g}_{i} \cdot ( 1 + λ \cdot {I}_{DEG} ( i ) ) ,$$

where I_{DEG}(i) is an indicator function that has a value of one when the node i is the DEG and has a value of zero when the node i is not the DEG, and λ is a weight constant for emphasizing the DEG. In this embodiment, the sample-level vector hₛ is calculated as

$${h}_{s} = \frac{{\sum }_{i ∈ {v}_{g}} {g}_{i}^{′} {h}_{i}}{{\sum }_{i ∈ {v}_{g}} {g}_{i}^{′}} .$$

In sub-step S043, the processor 11 makes the attention weight for the node of the heterogeneous graph serve as the gene-node contribution score. In one embodiment, the attention weight is an average of a k number of attention weights across k-fold cross-validation, where k is a positive integer. This way of determining the gene-node contribution score stands for a forward pass process of inference. In this embodiment, k is five, but is not limited thereto.

In one embodiment, the processor 11 determines the gene-node contribution score by using a gradient-based attribution method based on the feature vectors of the nodes of the heterogeneous graph. This way of determining the gene-node contribution score stands for a backward pass process of inference. Specifically, in mathematics, for a node i that represents one of the candidate genes and that has the feature vector

$${x}_{i}^{g}$$

which has D dimensions (for example, the feature vector

$${x}_{i}^{g} = [ \begin{matrix}{x}_{i}^{expr} \\ {x}_{i}^{deg} \\ {x}_{i}^{mut} \\ {x}_{i}^{path}\end{matrix} ]$$

has four dimensions, i.e., D=4), when the HGT determines in the forward pass process of inference that a probability of having the specific disease is P, the gene-node contribution score Sᵢ for the node i that represents one of the candidate genes is calculated in the backward pass process of inference according to the following formula:

$${S}_{i} = {\sum }_{d = 1}^{D} \vert \frac{\partial P}{\partial {x}_{i , d}} \vert .$$

It should be noted that the order of executing a set of steps S01 and S02 and a set of steps S03 and S04 is not limited to the disclosure herein and may vary in other embodiments. For example, in one embodiment, execution of the set of steps S01 and S02 is conducted in parallel with execution of the set of steps S03 and S04. In one embodiment, execution of the set of steps S03 and S04 is conducted prior to execution of the set of steps S01 and S02.

In step S05, the processor 11 generates, based on the pieces of target knowledge data included in the target knowledge database and the gene-node contribution scores respectively for the candidate genes, a target integrated evaluation table that contains multiple comprehensive assessment scores related respectively to the candidate genes.

Specifically, for each of the candidate genes, the processor 11 performs standardization on the tissue specificity score, the disease association score, the pathway association score, the cell essentiality score and the gene-node contribution score, so as to obtain a standardized tissue specificity score, a standardized disease association score, a standardized pathway association score, a standardized cell essentiality score and a standardized gene-node contribution score from the tissue specificity score, the disease association score, the pathway association score, the cell essentiality score and the gene-node contribution score, respectively, and then the processor 11 calculates a sum of the standardized tissue specificity score, the standardized disease association score, the standardized pathway association score, the standardized cell essentiality score and the standardized gene-node contribution score as the comprehensive assessment score for the candidate gene. Thereafter, the processor 11 sorts the comprehensive assessment scores respectively for the candidate genes in one of an ascending order and a descending order. In this embodiment, the comprehensive assessment scores are sorted in the descending order. In this embodiment, each of the standardized tissue specificity score, the standardized disease association score, the standardized pathway association score, the standardized cell essentiality score and the standardized gene-node contribution score ranges from zero to one. It should be noted that the way of performing standardization on the tissue specificity score, the disease association score, the pathway association score, the cell essentiality score and the gene-node contribution score is not limited to the disclosure herein and may vary in other embodiments.

In step S06, the processor 11 selects one of the candidate genes that corresponds to a greatest one of the comprehensive assessment scores in the target integrated evaluation table as the druggable target gene.

In step S07, the processor 11 outputs, via the communication module 12, the druggable target gene to a remote machine (not shown) to enable the remote machine to produce, based on the druggable target gene, a drug for the specific disease.

It should be noted that prior to using the HGT to obtain the gene-node contribution score for each of the candidate genes, the processor 11 trains the HGT by using supervised learning. Multiple pieces of training data correspond respectively to multiple samples (each of which may be a patient with the specific disease or a normal person) are prepared in advance. Each of the pieces of training data contains data required to construct a heterogeneous graph, and contains an expected result that is ground truth as to whether or not the corresponding one of the samples has the specific disease. Referring to FIG. 6, a procedure of training the HGT is illustrated. The procedure of training the HGT includes steps S11 to S16 delineated below.

In step S11, for each of the pieces of training data, the processor 11 first constructs a heterogeneous graph for training based on the piece of training data. Since step S11 in FIG. 6 is similar to step S03 in FIG. 2, detailed explanation of the same is omitted herein for the sake of brevity. Then, each of the heterogeneous graphs for training constructed respectively based on the pieces of training data is used as an input to the HGT stored in the storage 13, details of operations of the HGT are explained in steps S12 to S14.

In step S12, the HGT performs message passing with multi-head attention to update the feature vectors respectively of the nodes of the heterogeneous graph for training. Since step S12 in FIG. 6 is similar to sub-step S041 in FIG. 5, detailed explanation of the same is omitted herein for the sake of brevity.

In step S13, after the feature vectors respectively of the nodes of the heterogeneous graph have been updated, the processor 11 performs attention-based pooling to integrate the feature vectors into a sample-level vector. Since step S13 in FIG. 6 is similar to sub-step S042 in FIG. 5, detailed explanation of the same is omitted herein for the sake of brevity.

In step S14, the processor 11 uses the sample-level vector as an input to a linear classifier to obtain a prediction result. In mathematics, a process of converting the sample-level vector into the prediction result by using the linear classifier can be expressed as ŷ=W_{c}hₛ+b_{c}, where hₛ represents the sample-level vector, W_{c} is a learnable weight matrix, b_{c} is a learnable offset vector, and ŷ represents the prediction result (i.e., a result of classification by the linear classifier). It is worthy of note that the prediction result has not been processed by using a softmax function or a sigmoid function. In this embodiment, the prediction result is binary, and is exemplarily whether or not the specific disease occurs.

In step S15, after each of the heterogeneous graphs for training has been fed into the HGT, the processor 11 calculates a prediction probability of the HGT based on the prediction results obtained respectively for the heterogeneous graphs for training and the expected results contained respectively in the pieces of training data. The prediction probability is a probability that each of the prediction results correctly matches the corresponding one of the expected results (i.e., the ground truth). In other words, the prediction probability is a probability that the HGT correctly predicts whether or not each of the samples is the patient of the specific disease.

In step S16, the processor 11 adjusts parameters of the HGT based on the prediction probability according to a loss function. In this embodiment, the loss function is a focal loss function, but is not limited thereto. In mathematics, the focal loss function [symbol] is expressed as [symbol] =−αₜ(1−pₜ)^{y} log(pₜ), where pₜ is the prediction probability calculated in step S15, γ is a focusing parameter for making the HGT focus on samples that are difficult to be classified, αₜ is a hyperparameter that is determined according to a ratio of positives (i.e., a part of the samples that correspond to patients having the specific disease) to negatives (i.e., another part of the samples that correspond to normal people) for the pieces of training data. The ratio of positives to negatives can be determined according to the expected results (i.e., the ground truth) contained in the pieces of training data. The hyperparameter at is designed for alleviating issues caused by the ratio of positives to negatives that is extremely large or extremely small.

In order to verify the effectiveness of the method of identifying a druggable target gene according to the disclosure, experiments are conducted. Experimental data is obtained from the TCGA database, and includes transcriptomic data and genomic mutation data. The experimental data is used to construct heterogeneous graphs for the experiments. In particular, 20% of the training data serves as an independent test set, and is not used to train the HGT and is only used for evaluating performance of the HGT; 80% of the training data is used to conduct 5-fold cross-validation, and is performed with stratified splitting to ensure that training and testing sets have the same proportion of classes (or labels) for experimental (i.e., related to the specific disease) and control (i.e., unrelated to the specific disease) groups as original dataset (i.e., the 80% of the training data). Herein, evaluation metrics include precision, recall, F1-score, PR-AUC (area under precision-recall curve), and AUROC (area under receiver operating characteristic, ROC, curve).

A result of conducting 5-fold cross-validation is shown in Table 1 below. Since F1-scores respectively for Fold 1 to Fold 5 are all greater than 0.97, it is apparent that the method of identifying a druggable target gene according to the disclosure can effectively integrate the multi-omics data into the structure of heterogeneous graphs.

TABLE 1

| | Precision | Recall | F1-score | PR-AUC | AUROC |
|---|---|---|---|---|---|
| Fold 1 | 0.9904 | 0.9897 | 0.9900 | 0.9995 | 0.9941 |
| Fold 2 | 0.9977 | 0.9764 | 0.9870 | 0.9996 | 0.9954 |
| Fold 3 | 0.9933 | 0.9816 | 0.9874 | 0.9995 | 0.9942 |
| Fold 4 | 0.9985 | 0.9602 | 0.9790 | 0.9995 | 0.9944 |
| Fold 5 | 0.9992 | 0.9536 | 0.9759 | 0.9996 | 0.9954 |
| Average | 0.9955 | 0.9723 | 0.9839 | 0.9995 | 0.9947 |
| Standard | 0.0038 | 0.0150 | 0.0061 | 0.0001 | 0.0006 |
| deviation | | | | | |

A result of evaluation based on the independent test set is shown in Table 2 below. It is clear that the method of identifying a druggable target gene according to the disclosure could make accurate predictions for unknown samples, and thus has relatively good generalization ability.

TABLE 2

| | Average | Standard deviation |
|---|---|---|
| Precision | 0.9933 | 0.0027 |
| Recall | 0.9677 | 0.0129 |
| F1-score | 0.9803 | 0.0056 |
| PR-AUC | 0.9991 | 0.0002 |
| AUROC | 0.9900 | 0.0019 |

An example of the target integrated evaluation table obtained in the method according to the disclosure is shown in Tables 3A and 3B below, where a druggable target gene for KICH is to be identified. Since the candidate gene “mTOR” corresponds to a greatest one of the comprehensive assessment scores in the target integrated evaluation table, the candidate gene “mTOR” would be selected as the druggable target gene for KICH. In view of facts that mTOR inhibitors like Temsirolimus and Everolimus have been approved by U.S. Food and Drug Administration (FDA) for treating KICH, accuracy of predictions made by the method according to the disclosure may be affirmed. Moreover, candidate genes other than “mTOR”, especially those having high gene-node contribution scores in the target integrated evaluation table may have a high potential to serve as a target for developing new drugs for treating KICH.

TABLE 3A

| | | Tissue | Disease |
|---|---|---|---|
| | Gene-node | specificity | association |
| Gene | contribution score | score | score |
| mTOR | 0.46 | 0.58 | 0.62 |
| HNF1B | 0.48 | 0.85 | 0.64 |
| TP53 | 0.46 | 0.45 | 1.00 |
| ATM | 0.46 | 0.59 | 0.52 |
| FOXO1 | 0.45 | 0.61 | 0.35 |
| KMT2C | 0.45 | 0.49 | 0.52 |
| TSC1 | 0.46 | 0.48 | 0.52 |
| CDC42 | 0.46 | 0.58 | 0.00 |
| MAF | 0.46 | 0.53 | 0.52 |
| HNF1A | 0.50 | 0.56 | 0.71 |

TABLE 3B

| | Pathway | Cell | comprehensive | |
|---|---|---|---|---|
| | association | essentiality | assessment | |
| Gene | score | score | score | Rank |
| mTOR | 0.90 | 0.36 | 2.91 | 1 |
| HNF1B | 0.32 | 0.36 | 2.64 | 2 |
| TP53 | 0.69 | 0.00 | 2.60 | 3 |
| ATM | 0.76 | 0.13 | 2.46 | 4 |
| FOXO1 | 0.87 | 0.17 | 2.44 | 5 |
| KMT2C | 0.83 | 0.15 | 2.43 | 6 |
| TSC1 | 0.82 | 0.14 | 2.42 | 7 |
| CDC42 | 0.99 | 0.38 | 2.41 | 8 |
| MAF | 0.73 | 0.17 | 2.41 | 9 |
| HNF1A | 0.48 | 0.15 | 2.40 | 10 |

To sum up, for the method of identifying a druggable target gene according to the disclosure, the target knowledge database that includes multiple pieces of target knowledge data related respectively to multiple candidate genes is established based on multi-omics data, and at the same time, multiple gene-node contribution scores respectively for the candidate genes are obtained by using a heterogeneous graph as an input to the HGT. Thereafter, the target integrated evaluation table that contains multiple comprehensive assessment scores related respectively to the candidate genes is generated based on the pieces of target knowledge data and the gene-node contribution scores, and one of the candidate genes that corresponds to a greatest one of the comprehensive assessment scores in the target integrated evaluation table as the druggable target gene. Using multi-omics data in the method according to the disclosure may make identification of the druggable target gene relatively accurate and reliable. By virtue of multi-head attention in operations of the HGT, the gene-node contribution scores obtained by using the HGT may be explainable in a biological sense. Since identification of target genes and evaluation of druggability is implemented under a single framework, those target genes without druggability may be efficiently filtered out, thereby shortening a period of drug development.

In the description above, for the purposes of explanation, numerous specific details have been set forth in order to provide a thorough understanding of the embodiment(s). It will be apparent, however, to one skilled in the art, that one or more other embodiments may be practiced without some of these specific details. It should also be appreciated that reference throughout this specification to “one embodiment,” “an embodiment,” an embodiment with an indication of an ordinal number and so forth means that a particular feature, structure, or characteristic may be included in the practice of the disclosure. It should be further appreciated that in the description, various features are sometimes grouped together in a single embodiment, figure, or description thereof for the purpose of streamlining the disclosure and aiding in the understanding of various inventive aspects; such does not mean that every one of these features needs to be practiced with the presence of all the other features. In other words, in any described embodiment, when implementation of one or more features or specific details does not affect implementation of another one or more features or specific details, said one or more features may be singled out and practiced alone without said another one or more features or specific details. It should be further noted that one or more features or specific details from one embodiment may be practiced together with one or more features or specific details from another embodiment, where appropriate, in the practice of the disclosure.

While the disclosure has been described in connection with what is (are) considered the exemplary embodiment(s), it is understood that this disclosure is not limited to the disclosed embodiment(s) but is intended to cover various arrangements included within the spirit and scope of the broadest interpretation so as to encompass all such modifications and equivalent arrangements.

What is claimed is:

1. A method of identifying a druggable target gene for a specific disease from among multiple candidate genes, comprising steps of:
establishing, based on multi-omics data, a target knowledge database that includes multiple pieces of target knowledge data related respectively to the candidate genes;
constructing a heterogeneous graph related to the specific disease;
obtaining, for each of the candidate genes, a gene-node contribution score by using the heterogeneous graph as an input to a heterogeneous graph transformer (HGT);
generating, based on the pieces of target knowledge data included in the target knowledge database and the gene-node contribution scores respectively for the candidate genes, a target integrated evaluation table that contains multiple comprehensive assessment scores related respectively to the candidate genes; and
selecting one of the candidate genes that corresponds to a greatest one of the comprehensive assessment scores in the target integrated evaluation table as the druggable target gene.

2. The method as claimed in claim 1, wherein each of the pieces of target knowledge data includes a tissue specificity score that indicates an expression distribution of a corresponding one of the candidate genes in a specific tissue, a disease association score that quantifies a level of association between the corresponding one of the candidate genes and the specific disease, a pathway association score that indicates an impact of the corresponding one of the candidate gene on specific biological signaling pathways, and a cell essentiality score that indicates an effect of inhibiting the corresponding one of the candidate genes on cell survival and cell proliferation.

3. The method as claimed in claim 2, wherein the tissue specificity score is obtained by performing standardization on a ratio of a local expression level of the corresponding one of the candidate genes in the specific tissue to an average expression level of the corresponding one of the candidate genes in all tissues.

4. The method as claimed in claim 2, wherein the disease association score is an association score between the corresponding one of the candidate genes and the specific disease obtained from the Open Targets Platform.

5. The method as claimed in claim 2, wherein the pathway association score is obtained by determining multiple specific biological signaling pathways that are related to the corresponding one of the candidate genes, performing enrichment analysis on the specific biological signaling pathways to obtain p-values respectively of the specific biological signaling pathways, converting the p-values respectively into z-values as participation scores of the corresponding one of the candidate genes, and calculating an arithmetic average of the participation scores of the candidate gene as the pathway association score of the corresponding one of the candidate genes.

6. The method as claimed in claim 2, wherein the cell essentiality score is obtained by multiplying a gene effect score from the DepMap Portal database by minus one.

7. The method as claimed in claim 2, wherein the step of generating the target integrated evaluation table includes, for each of the candidate genes:
performing standardization on the tissue specificity score, the disease association score, the pathway association score, the cell essentiality score and the gene-node contribution score, so as to obtain a standardized tissue specificity score, a standardized disease association score, a standardized pathway association score, a standardized cell essentiality score and a standardized gene-node contribution score from the tissue specificity score, the disease association score, the pathway association score, the cell essentiality score and the gene-node contribution score, respectively; and
calculating a sum of the standardized tissue specificity score, the standardized disease association score, the standardized pathway association score, the standardized cell essentiality score and the standardized gene-node contribution score as the comprehensive assessment score for the candidate gene.

8. The method as claimed in claim 7, wherein each of the standardized tissue specificity score, the standardized disease association score, the standardized pathway association score, the standardized cell essentiality score and the standardized gene-node contribution score ranges from zero to one.

9. The method as claimed in claim 1, wherein the step of constructing the heterogeneous graph includes sub-steps of:
adding multiple nodes respectively representing the candidate genes, multiple nodes respectively representing candidate drugs for the specific disease, and multiple edges to the heterogeneous graph;
for each of the nodes that represent the candidate genes, initializing a feature vector of the node based on a piece of node-feature data related to a corresponding one of the candidate genes;
for each of the nodes that represent the candidate drugs, initializing a feature vector of the node based on a piece of node-feature data related to a corresponding one of the candidate drugs; and
for each of the edges, performing relation embedding on the edge with a relational descriptor that is related to a function type based on a piece of edge-feature data that is related to a protein-protein interaction (PPI) or a drug-target interaction (DTI).

10. The method as claimed in claim 9, wherein the feature vector of the node that represents one of the candidate genes includes an expression level of a transcriptome related to the one of the candidate genes, a gene-mutation indicator indicating whether or not a gene mutation occurs on the one of the candidate genes, a DEG indicator indicating whether or not the one of the candidate genes is a differentially expressed gene (DEG), and a signaling-pathway indicator indicating whether or not the one of the candidate genes is involved in a biological signaling pathway relevant to the specific disease.

11. The method as claimed in claim 9, wherein the feature vector of the node that represents one of the candidate drugs includes a chemical fingerprint that profiles compounds of the one of the candidate drugs, and a therapeutic indicator indicating whether or not the one of the candidate drugs has a therapeutic effect on the specific disease.

12. The method as claimed in claim 9, wherein the function type of the edge indicated by the relational descriptor is one of an adjustment, a physical action and a clinical effect.

13. The method as claimed in claim 9, wherein the step of obtaining the gene-node contribution score includes sub-steps of:
performing, by using the HGT, message passing with multi-head attention to update the feature vectors respectively of the nodes of the heterogeneous graph; and
after the feature vectors respectively of the nodes of the heterogeneous graph have been updated, performing attention-based pooling to integrate the feature vectors into a sample-level vector.

14. The method as claimed in claim 13, wherein the sub-step of performing attention-based pooling is to determine multiple attention weights respectively for the nodes of the heterogeneous graph, to calculate multiple weighted vectors by multiplying the feature vectors respectively by the attention weights, and to calculate an average of the weighted vectors as the sample-level vector.

15. The method as claimed in claim 14, wherein the step of obtaining the gene-node contribution score further includes sub-steps of:
making the attention weight for the node of the heterogeneous graph serve as the gene-node contribution score or determining the gene-node contribution score by using a gradient-based attribution method based on the feature vectors of the nodes of the heterogeneous graph.

16. The method as claimed in claim 13, further comprising a step of training the HGT that includes:
using the sample-level vector as an input to a linear classifier to obtain a prediction result;
calculating a prediction probability of the HGT at least based on the prediction result and an expected result; and
adjusting parameters of the HGT based on the prediction probability according to a loss function.

17. The method as claimed in claim 16, wherein the loss function is a focal loss function.

18. The method as claimed in claim 13, wherein the sub-step of performing message passing includes, for an edge that is between a current node i and a neighbor node j and that is embedded with a relational descriptor eᵢⱼ, with respect to an attention head h of the multi-head attention at a layer l of the HGT, each of i, j, h and l being an integer:
calculating a query vector
$${q}_{i}^{h} = {W}_{Q}^{h} {x}_{i} ,$$
where
$${W}_{Q}^{h}$$
is a learnable linear weight matrix for the attention head h, and xᵢ is the feature vector of the current node i;
calculating a key vector
$${k}_{j}^{h} = {W}_{K}^{h} {x}_{j} + {U}_{K}^{h} {e}_{ij} ,$$
where
$${W}_{K}^{h}$$
is a learnable linear weight matrix for the attention head h, xⱼ is the feature vector of the current node j, and
$${U}_{K}^{h}$$
is a learnable parameter linear weight matrix for the attention head h;
calculating a value vector
$${v}_{j}^{h} = {W}_{V}^{h} {x}_{j} + {U}_{V}^{h} {e}_{ij} ,$$
where
$${W}_{V}^{h}$$
is a learnable linear weight matrix for the attention head h, and
$${U}_{V}^{h}$$
is a learnable parameter;
calculating, by using a softmax function, an attention score
$${α}_{ij}^{h} = \frac{exp ( \frac{{( {q}_{i}^{h} )}^{T} {k}_{j}^{h}}{\sqrt{{d}_{k}}} )}{{\sum }_{j ∈ N ( i )} exp ( \frac{{( {q}_{i}^{h} )}^{T} {k}_{j}^{h}}{\sqrt{{d}_{k}}}} ,$$
where dₖ is a scaling factor;
calculating, for the current node i for the attention head h, an information-aggregation vector
$${m}_{i}^{h} = {\sum }_{j ∈ N ( i )} {α}_{ij}^{h} {v}_{j}^{h} ;$$
and
performing residual connection and layer normalization on
$${h}_{i}^{l} ,$$
which is an original feature vector of the current node i at the layer l of the HGT, to obtain
$${h}_{i}^{l + 1} = LayerNorm ( ReLU ( {m}_{i} ) + {W}_{res} {h}_{i}^{l} ) ,$$
which is an updated feature vector of the current node i at a layer l+1 of the HGT, where mᵢ is a result of residual connection performed for all attention heads, Wᵣₑₛ is a residual projection matrix, ReLU( ) is a rectified linear unit activation function, and LayerNorm( ) is a layer normalization function.

19. The method as claimed in claim 1, wherein the step of generating the target integrated evaluation table includes sorting the comprehensive assessment scores in one of an ascending order and a descending order.

20. The method as claimed in claim 1, further comprising a step of training the HGT by using supervised learning.
